(19) United States
(12) Patent Application Publication
(10) Pub. No.: US 2012/0296138 A1
(43) Pub. Date: Nov. 22, 2012

(54) METHOD FOR ALKYLATION OF TOLUENE TO FORM STYRENE AND ETHYLBENZENE UTILIZING A MOVING BED REACTOR

(75) Inventor: Joseph E. Pelati, Houston, TX (US)

(73) Assignee: FINA TECHNOLOGY, INC., Houston, TX (US)

(21) Appl. No.: 13/457,502

(22) Filed: Apr. 27, 2012

Related U.S. Application Data
(60) Provisional application No. 61/488,773, filed on May 22, 2011.

(51) Int. Cl. C07C 2/88; B01J 8/02

(52) U.S. Cl. 585/437; 422/211; 422/223

(57) ABSTRACT

A method is disclosed for making styrene and/or ethylbenzene by reacting toluene with a C₁ source in the presence of a catalyst in a reactor sized and configured to provide for a moving catalyst bed therethrough to form a product stream including styrene and/or ethylbenzene.

### CROSS-REFERENCE TO RELATED APPLICATIONS

This application claims priority to U.S. Provisional Patent No. 61/488,773 filed on May 22, 2011.

### FIELD

The present invention relates to a method for the production of styrene and ethylbenzene. More specifically, the invention relates to a method for the alkylation of toluene with methanol and/or formaldehyde to produce styrene and ethylbenzene utilizing a moving bed reactor with a heterogeneous catalyst.

### BACKGROUND

Styrene is an important monomer used in the manufacture of many plastics. Styrene is commonly produced by making ethylbenzene, which is then dehydrogenated to produce styrene. Ethylbenzene is typically formed by one or more aromatic conversion processes involving the alkylation of benzene.

Aromatic conversion processes, which are typically carried out utilizing a molecular sieve type catalyst, are well known in the chemical processing industry. Such aromatic conversion processes include the alkylation of aromatic compounds such as benzene with ethylene to produce alkyl aromatics such as ethylbenzene. Typically an alkylation reactor, which can produce a mixture of monoalkyl and polyalkyl benzenes, will be coupled with a transalkylation reactor for the conversion of polyalkyl benzenes to monoalkyl benzenes. The transalkylation process is operated under conditions to cause disproportionation of the polyalkylated aromatic fraction, which can produce a product having an enhanced ethylbenzene content and reduced polyalkylated content. When both alkylation and transalkylation processes are used, two separate reactors, each with its own catalyst, can be employed for each of the processes.

Ethylene is obtained predominantly from the thermal cracking of hydrocarbons, such as ethane, propane, butane, or naphtha. Ethylene can also be produced and recovered from various refinery processes. Thermal cracking and separation technologies for the production of relatively pure ethylene can account for a significant portion of the total ethylbenzene production costs.

Benzene can be obtained from the hydrodealkylation of toluene that involves heating a mixture of toluene with excess hydrogen to elevated temperatures (for example 500° C. to 600° C.) in the presence of a catalyst. Under these conditions, toluene can undergo dealkylation according to the chemical equation: C₆H₅CH₃+H₂→C₆H₆+CH₄ This reaction requires energy input and as can be seen from the above equation, produces methane as a byproduct, which is typically separated and may be used as heating fuel for the process.

Another process includes the alkylation of toluene to produce styrene and ethylbenzene. In this alkylation process, various catalysts can be utilized to react methanol and toluene to produce styrene and ethylbenzene. It has been found in the alkylation of toluene to produce styrene and ethylbenzene that an increase in contact time between the reactants and catalysts increases the likelihood of undesired reactions in the reactor, including the hydrogenation of styrene to ethylbenzene. Known solutions in the art to decrease contact time vary depending on the reactor, reactants, and catalyst desired. For example, in a fixed bed reactor, the catalyst bed may be reduced in thickness in order to reduce contact time. However, such a reduction in thickness or bed size typically results in a shorter catalyst onstream time and the frequency of replacement or regeneration of the catalyst is increased. Another known solution in the art to reduce contact time is the use of a fluidized bed reactor. However, a fluidized bed reactor requires the feedstream to be fed into the fluidized bed reactor at a high flow rate in order to sufficiently agitate and transport the catalyst. Reaction kinetics, energy and equipments costs associated with producing the high flow rate may be economically undesirable in certain circumstances.

In view of the above, it would be desirable to have a process of producing styrene and/or ethylbenzene that does not rely on thermal crackers and expensive separation technologies as a source of ethylene. It would further be desirable to avoid the process of converting toluene to benzene with its inherent expense and loss of a carbon atom to form methane. It would be desirable to produce styrene without the use of benzene and ethylene as feedstreams. It would also be desirable to produce styrene with minimal contact time to increase the efficiency of the production of styrene and to reduce undesirable reactions, such as hydrogenation of styrene to ethylbenzene. Furthermore, it is desirable to have a reactor system able to achieve a high yield and selectivity to styrene and ethylbenzene.

### SUMMARY

The present invention in its many embodiments relates to a method of making styrene utilizing a reactor having a moving catalyst bed.

An embodiment of the present invention, either by itself or in combination with other embodiments, is a method is provided for making styrene including reacting toluene with a C₁ source in a reactor in the presence of a catalyst to form a product stream including ethylbenzene and styrene. The reactor is sized and configured to provide for at least one moving catalyst bed therethrough. The C₁ source is selected from the group that consists of methanol, formaldehyde, formalin, trioxane, methylformcel, paraformaldehyde, methylal, dimethyl ether, and combinations thereof.

In an embodiment, either by itself or in combination with any other embodiment, the reactor includes a shell defining an interior cavity and at least one interior member defining a plurality of member openings including at least one particulate opening and at least one fluid opening. The interior member is disposed within the interior cavity and sized and configured to temporarily retain at least a portion of the catalyst and to permit at least a portion of a feedstream including the toluene and C₁ source to flow through the fluid opening and react over the catalyst, thereby forming at least one spent catalyst. The particulate opening can be sized and configured to allow a flow of catalyst particulates therethrough and the fluid opening can be sized and configured to allow the feedstream to flow therethrough and to create turbulence in the feedstream such that the turbulent feedstream agitates and displaces at least a portion of the temporarily retained catalyst.

In an embodiment, either by itself or in combination with any other embodiment, the catalyst is based on a zeolite selected from the group consisting of faujasites. Optionally, the catalyst is based on an X-type zeolite. The catalyst can be promoted with a promoter selected from the group consisting of Co, Mn, Ti, Zr, V, Nb, K, Cs, Ga, B, P, Rb, Ag, Na, Cu, Mg, Fe, Mo, Ce, and combinations thereof.

The method can further include withdrawing the product stream and spent catalyst from the reactor; regenerating the spent catalyst in a regeneration vessel to produce regenerated catalyst; and recycling the regenerated catalyst to the reactor. The catalyst time on stream prior to regeneration can be less than 24 hours. The contact time of the reactants with the catalyst in the reactor can be less than 10 seconds.

The various embodiments of the present invention can be joined in combination with other embodiments of the invention and the listed embodiments herein are not meant to limit the invention. All combinations of various embodiments of the invention are enabled, even if not given in a particular example herein.

### BRIEF DESCRIPTION OF DRAWINGS

FIG. 1 illustrates a cross-sectional view of a reactor consistent with one embodiment of the present invention, wherein the reactor includes a plurality of trays, wherein each tray defines a plurality of openings sized and configured to allow catalyst particulates and gas to flow through the reactor with different flow rates.

FIG. 2 illustrates a cross-sectional view of a reactor consistent with an alternate embodiment of the present invention, wherein the reactor is sized and configured to provide for a moving bed therethrough and is operating in the dilute phase when coupled to the systems illustrated in FIGS. 4 and 4A below.

FIG. 2A illustrates a cross-sectional view of a reactor consistent with an alternate embodiment of the present invention, wherein the reactor is sized and configured to provide for a moving bed therethrough and is operating in the dense phase when coupled to the system illustrated in FIG. 4 below.

FIG. 3 illustrates a flow diagram of a process consistent with one embodiment of the present invention, wherein the process is carried out in a counter-current flow direction.

FIG. 4 illustrates a flow diagram of a process consistent with an alternate embodiment of the present invention, wherein the process is carried out in a concurrent flow direction.

FIG. 4A illustrates a flow diagram of a process consistent with an alternate embodiment of FIG. 4, wherein the process is carried out in a concurrent flow direction in a direction opposing the flow direction of FIG. 4.

### DETAILED DESCRIPTION

In an embodiment of the current invention, a process includes the utilization of a moving bed reactor sized and configured to provide for at least one moving bed therethrough, wherein the moving bed reactor includes a shell defining an interior cavity. The moving bed reactor can include at least one interior member defining a plurality of member openings, wherein the interior member is disposed within the interior cavity and sized and configured to temporarily retain at least a portion of a catalyst and to permit at least a portion of a feedstream to flow through at least a portion of the member openings, such that the feedstream may flow through the moving bed reactor and contact the catalyst. In one embodiment, the feedstream includes toluene and a carbon (C₁) source.

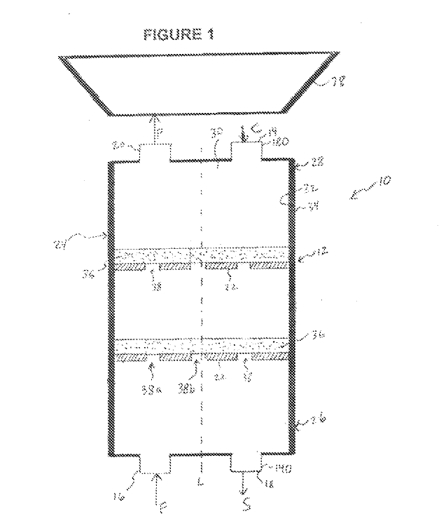

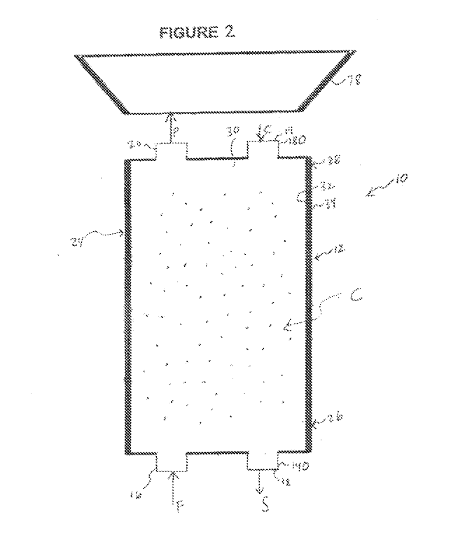

Turning now to the Figures, FIGS. 1, 2, and 2A each illustrate an embodiment of a moving bed reactor (10) of the present invention. The moving bed reactor (10) includes an elongated cylindrical shell (12) that includes a catalyst inlet (14), a feedstream inlet (16), a catalyst outlet (18), and a feedstream outlet (20). As shown in FIG. 1, the moving bed reactor can contain a plurality of interior members (22). In FIGS. 1, 2, and 2A, the elongated cylindrical shell (12) further comprises a vertical cylindrical sidewall (24) connecting a feedstream inlet side portion (26) defining the feedstream inlet (16) and a feedstream outlet side portion (28) defining the feedstream outlet (20). The vertical cylindrical sidewall (24) defines an interior cavity (30) and comprises an internal sidewall surface (32) and an external sidewall surface (34). The feedstream inlet side portion (26) defines the feedstream inlet (16) such that the feedstream inlet allows a feedstream (F) to be fed into the interior cavity (30). The feedstream outlet side portion (28) defines the feedstream outlet (20) such that the feedstream outlet allows a product stream (P) to exit from the interior cavity (30) of the moving bed reactor (10).

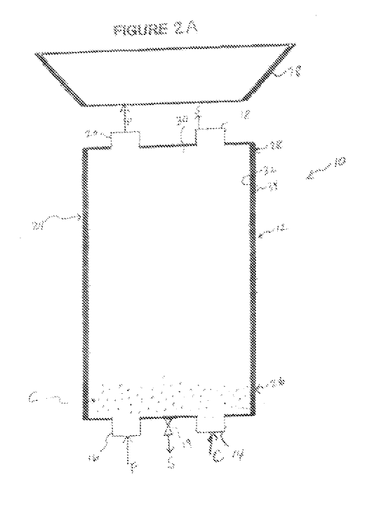

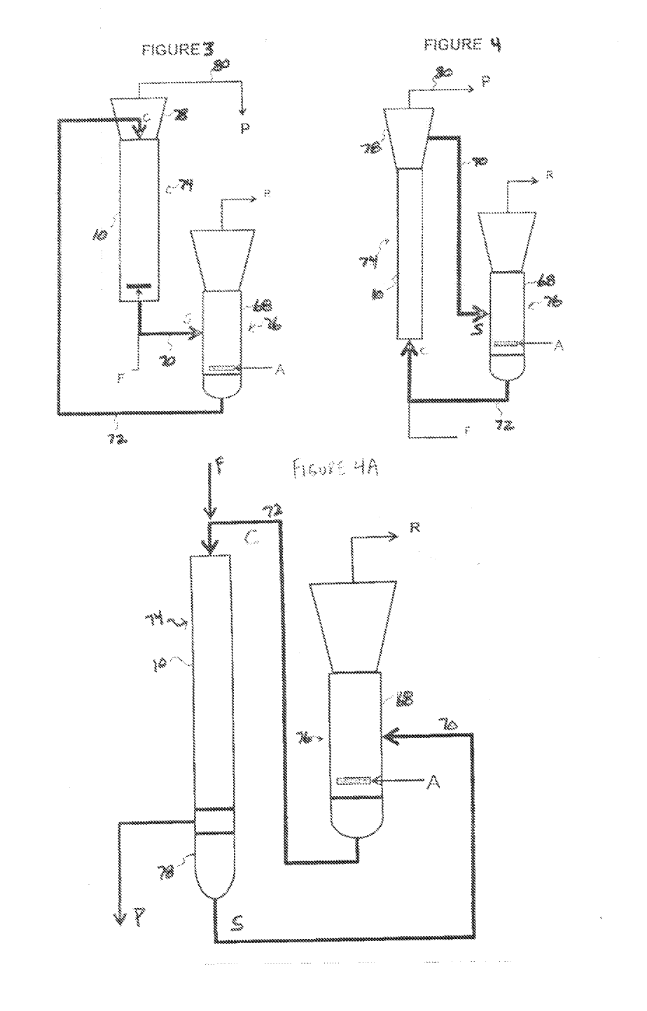

The feedstream inlet side portion and the feedstream outlet side portion further define the catalyst inlet and the catalyst outlet depending on the direction of flow in the moving bed reactor. As will be discussed below, the moving bed reactor will operate in either a counter-current flow direction as shown in FIGS. 1 and 3, or a concurrent flow direction as shown in FIGS. 2, 2A, 4 and 4A. The feedstream inlet side portion (26) defines the catalyst inlet (14) when the moving bed reactor operates in the concurrent flow direction and defines the catalyst outlet (18) when the moving bed reactor (10) operates in the counter-current flow direction. The feedstream outlet side portion (28) defines the catalyst outlet (18) when the moving bed reactor (10) operates in the concurrent flow direction and defines the catalyst inlet (14) when the moving bed reactor operates in the counter-current flow direction.

As shown in FIG. 1, the interior members (22) are trays disposed within the interior cavity (30), such that the trays have an upstream face and an opposing downstream face. The upstream face is orientated in the moving bed reactor (10) facing the feedstream inlet side portion (26) and the downstream face is orientated facing the feedstream outlet side portion (28) when the tray (22) is orientated substantially perpendicular to the longitudinal axis (L) of the moving bed reactor. Each of the trays (22) is designed to temporarily retain at least a portion of the catalyst material, whereby the temporarily retained catalyst material disposed on each tray forms a catalyst bed (36). In the embodiment illustrated in FIG. 1, the trays (22) are attached to the vertical cylindrical sidewall (24) and define a plurality of member openings (38). The plurality of member openings include at least one particulate opening (38a) and at least one fluid opening (38b), wherein the particulate opening is sized and configured to allow particulates to flow therethrough and the fluid opening is sized and configured to allow fluid (F) to flow therethrough. The fluid opening can be further configured to create turbulence in the fluid flow as the fluid flows therethrough, thus destabilizing the catalyst bed formed on a tray and causing the bed to become at least partially fluidized. The plurality of member openings can include a plurality of particulate openings and a plurality of fluid openings. A separation vessel (78) or cyclone can be used for a subsequent separation of any entrained solids in the product stream (P) exiting the reactor (10). The catalyst can exit as stream (S).

In at least one embodiment, the trays are attached by bolts and are formed from individual tray components, whereby individual tray components may be removed to allow access to various portions of the reactor. The type of tray used is system specific and can vary depending on the feedstream composition and the composition of the product streams. Nonlimiting examples of conventional trays that may be used include bubble cap, sieve, and valve trays.

In an alternate embodiment, the moving bed reactor includes movable trays. The moving bed reactor may have various means for moving the trays within the moving bed reactor. The trays may be moved along or parallel to the longitudinal axis of the moving bed reactor or along or parallel to the latitudinal axis of the reactor, or in a direction both along or parallel to the longitudinal and latitudinal axis of the moving bed reactor. Nonlimiting examples of means for moving the trays include vibration sources, drive belts, motors, pulleys, sprockets, chains, and the like.

In an alternate embodiment, an interior member forms a screw auger, wherein the screw auger is disposed within the interior cavity and is sized and configured to continuously transport at least a portion of the catalyst particulates through the moving bed reactor. The screw auger can be powered and rotated by an external power source such as a diesel or electric motor. Optionally, the screw auger may rotate from a fluid flow through the moving bed reactor.

In an alternate embodiment, the moving bed reactor is rotated by external means. For example, the moving bed reactor can be a rotary kiln, wherein the moving bed reactor is rotated by an external drive gear coupled to a drive train powered by an electric motor. Optionally, the moving bed reactor may be rotated by a diesel motor. The rotary kiln rotates about a longitudinal axis of the rotary kiln, thus effectively moving catalyst particulates fed through the rotary kiln and creating a continuously moving bed reactor.

In one embodiment, the feedstream includes toluene and a C₁ source. In an embodiment, the C₁ source includes methanol or formaldehyde or a mixture of the two. In an alternative embodiment, the C₁ source includes one or more of the following: formalin, trioxane, methylformcel, paraformaldehyde, methylal, and dimethyl ether. In a further embodiment, the C₁ source is selected from the group consisting of methanol, formaldehyde, dimethyl ether, formalin (37-50% H₂CO in solution of water and MeOH), trioxane (1,3,5-trioxane), methylformcel (55% H₂CO in methanol), paraformaldehyde and methylal (dimethoxymethane), and combinations thereof.

Formaldehyde can be produced either by the oxidation or dehydrogenation of methanol. In an embodiment, formaldehyde is produced by the dehydrogenation of methanol to produce formaldehyde and hydrogen gas. This reaction step produces a dry formaldehyde stream that may be preferred, as it would not require the separation of the water prior to the reaction of the formaldehyde with toluene. Formaldehyde can also be produced by the oxidation of methanol to produce formaldehyde and water.

In the case of using a separate process to obtain formaldehyde, a separation unit may then be used in order to separate the formaldehyde from the hydrogen gas or water from the formaldehyde and unreacted methanol prior to reacting the formaldehyde with toluene for the production of styrene. This separation will inhibit the hydrogenation of the formaldehyde back to methanol. Purified formaldehyde can then be sent to the moving bed reactor and the unreacted methanol can be recycled.

Turning now to the embodiment illustrated in FIG. 3, a system and process are provided for the production of styrene and ethylbenzene. In this embodiment, the process is operated in a counter-current flow direction and has the capability for continuous reaction with catalyst regeneration. In operation, the process utilizes a moving bed reactor (10), a regeneration vessel (68), and a spent catalyst conduit (70) and a regenerated catalyst conduit (72), wherein the spent catalyst conduit and the regenerated catalyst conduit each operatively connect the reactor and the regeneration vessel forming a catalyst flow loop. Generally, the process utilizes two main zones for reaction and regeneration. The two main zones include the reaction zone (74) and the regeneration zone (76). The reaction zone includes the moving bed reactor (10) as the main reaction site, with the effluent of the moving bed reactor emptying into a large volume process vessel, which may be referred to as a separation vessel (78) or cyclone where a product stream (P) can exit via line (80). The characteristics of the reaction zone vary depending on the moving bed reactor used in the process. The regeneration zone (76) can regenerate the catalyst (C) to be sent via regenerated catalyst conduit (72) to the reaction zone (74), and produce a residue stream (R).

Looking now at FIGS. 1 and 3, an embodiment of the system and process for the production of styrene is illustrated. A feedstream (F) including toluene and a C₁ source is fed into the feedstream inlet (16) defined by the feedstream inlet side portion (26) of the moving bed reactor (10) (the internal structure of the reactor shown in more detail in FIG. 1). Catalyst material (C) is fed through the catalyst inlet (14) defined by the feedstream outlet side portion (28) of the reactor (10). Thus, the feedstream (F) and the catalyst material (C) are fed into the reactor (10) in opposing flow directions, or in a counter-current flow direction.

The catalyst material (C), aided by gravitational force, flows downward onto the downstream face of the tray (22) proximate the catalyst inlet (14), wherein a portion of the catalyst material is temporarily retained, thus forming a catalyst bed (36). Another portion of the catalyst material (C) flows through the particulate openings (38a) onto the next tray (22) below the topmost tray, wherein a portion of the catalyst is temporarily retained on the tray, thus forming a catalyst bed. This process repeats itself as the catalyst flows downward through particulate openings in the respective trays. The feedstream (F) flows through the fluid openings (38b) in each tray (22) in an upward direction toward the feedstream outlet (20) and contacts the catalyst material (C) as it flows downward and also contacts the temporarily-formed catalyst bed (36) on each tray. The fluid openings are configured to create turbulence in the feedstream flowing through the fluid openings. The turbulent feedstream created provides the temporarily-formed catalyst bed with sufficient agitation to displace at least a portion of the bed and allows increased contacting of the catalyst particulates with the feedstream. Additionally, at least a portion of the displaced catalyst particulates can flow through the particulate openings downward to the next tray. As the feedstream (F) flows through the moving bed reactor (10) and contacts the catalyst (C), a product stream (P) including styrene is formed. The product stream (P) flows through the feedstream outlet (20) into the separation vessel (78). As the catalyst particulates deactivate from contact with the feedstream, the spent catalyst material (S) flows downward through the catalyst outlet (18) defined by the feedstream inlet side portion (26) through the spent catalyst conduit (70) into the regeneration vessel (68), wherein the catalyst is contacted with regeneration stream (A) and regenerated.

In the system and process of the embodiment illustrated in FIGS. 1 and 3, the contact time between the feedstream and the catalyst material ranges from 0.1 seconds to 10 seconds. Optionally, the contact time between the feedstream and the catalyst material ranges from 0.1 seconds to 5 seconds. Optionally, the contact time between the feedstream and the catalyst material is less than 1 second. The catalyst residence time in the system and process of the embodiment illustrated in FIGS. 1 and 3 is less than 24 hours. Optionally, the catalyst residence time ranges from 0.5 minutes to 1 hour. Optionally, the catalyst residence time ranges from 2 minutes to 4 minutes.

Referring now to FIGS. 4 and 4A, alternate embodiments of a system and process for producing styrene is illustrated. In these embodiments, the process is carried out in a concurrent flow direction and has the capability for continuous reaction with catalyst regeneration. In operation, the process utilizes a moving bed reactor (10), a regeneration vessel (68), and a spent catalyst conduit (70) and a regenerated catalyst conduit (72), wherein the spent catalyst conduit and the regenerated catalyst conduit each operatively connect the reactor and the regeneration vessel forming a catalyst flow loop. Generally, there are two main zones for reaction and regeneration. The two main zones include the reaction zone (74) and the regeneration zone (76). The reaction zone (74) includes the moving bed reactor (10) as the main reaction site, with the effluent of the moving bed reactor emptying into a large volume process vessel, which may be referred to as a separation vessel (78) where a product stream (P) can exit via line (80). The characteristics of the reaction zone vary depending on the moving bed reactor used in process. The regeneration zone (76) can regenerate the catalyst (C) to be sent via regenerated catalyst conduit (72) to the reaction zone (74), and produce a residue stream (R).

As disclosed above, the moving bed reactors illustrated in FIG. 2 and FIG. 2A can be employed in the system and process of the embodiments illustrated in FIGS. 4 and 4A, wherein the process is carried out in the concurrent flow direction. Generally, the moving bed reactors shown in FIGS. 2 and 2A can operate in the dilute phase and the dense phase respectively in the systems illustrated in FIGS. 4A and 4 respectively. The operation of the systems will depend at least in part on the gas flow velocity of the feedstream through the moving bed reactor and the size of the catalyst particulates fed to the moving bed reactor.

In operation in the dilute phase as illustrated in FIG. 2, the feedstream flows through the moving bed reactor with a gas flow velocity ranging from 0.1 to 3.0 m/s, optionally from 0.2 to 1.5 m/s, optionally from 0.5 to 1.5 m/s. The moving bed reactor may have a solid catalyst velocity ranging from 0.1 to 20 cm/s, optionally from 1 to 10 cm/s, optionally from 4 to 8 cm/s. The residence time for the gas stream within the reactor may range from 1-30 seconds, optionally from 2-20 seconds, optionally from 3-10 seconds. In an embodiment in the dilute phase operation the average catalyst particle diameter size can range from 1-300 microns, optionally from 5-200 microns, optionally from 10-150 microns.

In operation in the dense phase as illustrated in FIG. 2A, the feedstream flows through the moving bed reactor with a gas flow velocity ranging from 100 to 10,000 hr⁻¹ gas hourly space velocity GHSV. Optionally, the gas flow velocity of the feedstream through the moving bed reactor ranges from 250 to 6,000 hr⁻¹ GHSV, optionally from 400-1,000 hr⁻¹ GHSV. In an embodiment in the dense phase operation the average catalyst particle diameter size can range from 10 microns to 10 mm, optionally from 25 microns to 6 mm, optionally from 70 microns to 3 mm.

Accordingly, the higher gas flow velocity and the smaller catalyst particulate size in a moving bed reactor operating in the dilute phase allows the feedstream to carry the catalyst material through the moving bed reactor at a faster rate than the catalyst material in the moving bed reactor operating in the dense phase. The dilute phase provides for partially suspended catalyst material. In a moving bed reactor operating in the dense phase, the larger size of the catalyst material and the lower feedstream velocity creates a “bubbling bed,” wherein a dense bed is created with the feedstream flowing and “bubbling” therethrough. Thus, the catalyst residence time is greater in the moving bed reactor operating in the dense phase than in the dilute phase.

In a moving bed reactor operating in the dilute phase, the catalyst residence time ranges from 0.1 seconds to 10 seconds. Optionally, the catalyst residence time ranges from 0.1 seconds to 5 seconds. Optionally, the catalyst residence time is less than 1 second. The contact time between the feedstream and the catalyst material in the dilute phase ranges from 0.1 seconds to 10 seconds. Optionally, the contact time between the feedstream and the catalyst material ranges from 1 second to 5 seconds. Optionally, the contact time between the feedstream and the catalyst material is less than 2.5 seconds.

In a moving bed reactor operating in the dense phase, the catalyst residence time ranges from 1 minute to 3 weeks. Optionally, the catalyst residence time ranges from 2 hours to 2 weeks. Optionally, the catalyst residence time is less than 1 week. The contact time between the feedstream and the catalyst material in the dense phase ranges from 0.1 seconds to 10 seconds. Optionally, the contact time between the feedstream and the catalyst material ranges from 1 second to 5 seconds. Optionally, the contact time between the feedstream and the catalyst material is less than 2.5 seconds.

FIGS. 2A and 4 illustrate an alternate embodiment of the system and process for the production of styrene. A feedstream (F) including toluene and a C₁ source is fed into the feedstream inlet (16) defined by the feedstream inlet side portion (24) of the moving bed reactor (10) (the internal structure of the reactor shown in more detail in FIG. 2) operating in the dilute phase. Catalyst material (C) is fed through the catalyst inlet (14) defined by the feedstream inlet side portion (26) of the reactor (10). Thus, the feedstream and the catalyst material are fed into the reactor in a concurrent flow direction. The catalyst material (C) flows upward from the catalyst inlet (14) and contacts and is temporarily suspended in the feedstream (F) flowing upward through the feedstream inlet (16). The residence time of the catalyst particulates in the moving bed reactor can depend in part on the flow rate of the feedstream and the type and size of the catalyst particulates utilized.

Upon deactivation of the catalyst, the spent catalyst material (S) flowing upward through the reactor (10) flows through the catalyst outlet (18) defined by the feedstream outlet side portion (28) into the spent catalyst conduit (70) and into the regeneration vessel (68). The product stream (P) formed by the reactants flowing over the catalyst material (C) exits the moving bed reactor (10) through the feedstream outlet (20) into the separation vessel (78).

The moving bed reactor can operate in the dilute phase or the dense phase in the embodiment illustrated in FIG. 4 or 4A. The residence time of the catalyst particulates in the moving bed reactor can depend in part on the flow rate of the feedstream and the type and size of the catalyst particulates utilized.

Upon deactivation of the catalyst, the spent catalyst material (S) flowing upward through the reactor (10) in the feedstream (F) flows through the catalyst outlet (18) defined by the feedstream outlet side portion (28) into the spent catalyst conduit (70) and into the regeneration vessel (68). The product stream (P) formed by the reactants flowing over the catalyst material (C) exits the moving bed reactor (10) through the feedstream outlet (20) into the separation vessel (78).

Alternatively, the moving bed reactor (10) operates in the dense phase. A feedstream (F) including toluene and a C₁ source is fed into the feedstream inlet (16) defined by the feedstream inlet side portion (26) of the moving bed reactor (10) operating in the dense phase. Catalyst material (C) is fed through the catalyst inlet (14) defined by the feedstream inlet side portion (26) of the reactor (10). Thus, the feedstream and the catalyst material are fed into the reactor in a concurrent flow direction. The catalyst material (C) flows upward from the catalyst inlet (14) and accumulates in the feedstream inlet side portion (26). Feedstream (F) flowing upward through the feedstream inlet (16) contacts at least a portion of the catalyst material (C), wherein the feedstream velocity decreases as the feedstream flows through the catalyst material such that at least a portion of the feedstream forms at least one bubble as it flows, thereby creating a bubbling flow through the catalyst material. The residence time of the catalyst particulates in the moving bed reactor can depend in part on the flow rate of the feedstream and the type and size of the catalyst particulates utilized. Additionally, a removal device (19) can be employed at the bottom of the moving bed reactor to remove a portion of the spent catalyst (S) from the moving bed reactor and into the regeneration vessel (68). A nonlimiting example of a removal device is a gate valve. Another portion of the spent catalyst (S) can be carried out through the catalyst outlet (18) and into the spent catalyst conduit (70) and into the regeneration vessel (68). The product stream (P) formed by the reactants flowing over the catalyst material (C) exits the moving bed reactor (10) through the feedstream outlet (20) into the separation vessel (78).

The residence time of catalyst and hydrocarbons in the moving bed reactor (10) needed for substantial completion of the reaction may vary as needed for the specific reactor design and throughput design. As shown in FIGS. 3, 4, and 4A, the flowing product stream leaving the moving bed reactor (10) may pass from the reactor to a solids-vapor separation device, such as a cyclone (78). The products of the reaction can be separated from the portion of catalyst that is carried by the product stream by means of one or more cyclone and the products can exit the separation vessel (78) via a flow line (80). The spent catalyst falls downward to a stripper (not shown) located in a lower part of the separation vessel (78). Catalyst can be transferred to a regeneration vessel (68) by way of a conduit (70). The regenerator effluent is sent for further processing via line (R).

In an embodiment, the reactants may be injected into the reactor(s) in a stage-wise manner. The toluene feed may be injected at any point, or points, along the moving bed reactor. In the embodiment of FIGS. 1 and 3, the C₁ source and/or the toluene feed can be injected in between each tray (22) or at least one tray. The C₁ source, which may include formaldehyde, may also be injected at any point, or points, along the moving bed reactor. In an embodiment, the toluene feed is injected downstream from the C₁ source injection point. In another embodiment, the C₁ source is injected downstream from the toluene feed injection point. In a further embodiment, both the C₁ source and the toluene feed are injected at the same point along the moving bed reactor.

Although the reaction has a stoichiometric 1:1 molar ratio of toluene and the C₁ source, the ratio of the feedstreams is not limited within the present invention and can vary depending on operating conditions and the efficiency of the reaction system. If excess toluene or C₁ source is fed to the reaction zone, the unreacted portion can be subsequently separated and recycled back into the process. In one embodiment the ratio of toluene:C₁ source can range from between 100:1 to 1:100. In alternate embodiments the ratio of toluene:C₁ source can range between from 50:1 to 1:50; from 20:1 to 1:20; from 10:1 to 1:10; from 5:1 to 1:5; from 2:1 to 1:2.

The operating conditions of the reactors and separators can be system specific and can vary depending on the feedstream composition and the composition of the product streams. The moving bed reactor for the reaction of toluene and formaldehyde will operate at elevated temperatures and pressures and may contain a basic or neutral catalyst system. The temperature can range in a non-limiting example from 250° C. to 750° C., optionally from 300° C. to 500° C., optionally from 375° C. to 450° C. The partial pressure can range in a non-limiting example from 0.1 atm to 70 atm, optionally from 0.1 atm to 35 atm, optionally from 0.1 atm to 10 atm, optionally from 0.3 atm to 3 atm.

In the reaction zone of at least one embodiment, wherein the coupling reaction of toluene with a C₁ source such as formaldehyde occurs, the conversion of toluene has been observed to be highest at the earliest segment of the contact time between the fresh or regenerated catalyst and the reactants. The contact time of catalyst and hydrocarbons in the moving bed reactor needed for substantial completion of the reaction may vary as needed for the specific reactor design and throughput design. It has been determined that as the contact time increases, the likelihood of undesirable reactions increases, such as the hydrogenation of styrene to ethylbenzene. Thus, it is desirable to decrease the contact time in the present invention.

In one embodiment, the contact time is limited and the catalyst is continually being regenerated to enable the reaction to achieve a higher toluene conversion. In one embodiment a moving bed reactor type of design is utilized to enable a short contact time and period of use of the catalyst prior to regeneration and to allow for a regeneration step to take place in order to bring the catalyst back to a more active state for reuse in the reactor in a recycle mode configuration.

The catalytic reaction systems suitable for this invention can include one or more of a zeolite, other catalytic materials, or substrate modified for side chain alkylation selectivity. A non-limiting example can be a zeolite promoted with one or more of the following: Co, Mn, Ti, Zr, V, Nb, K, Cs, Ga, B, P, Rb, Ag, Na, Cu, Mg, Fe, Mo, Ce, or combinations thereof. In an embodiment, the zeolite can be promoted with one or more of Ce, Cu, P, Cs, B, Co, Ga, or combinations thereof.

Zeolite materials suitable for this invention may include silicate-based zeolites and amorphous compounds such as faujasite, mordenite, chabazite, offretite, clinoptilolite, erionite, sihealite, and the like. Silicate-based zeolites are made of alternating SiO₂ and MOₓ tetrahedra, where M is an element selected from the Groups 1 through 16 of the Periodic Table (new IUPAC). These types of zeolites have 4-, 6-, 8-, 10-, or 12-membered oxygen ring channels. An example of zeolites of this invention can include faujasites. Other suitable zeolite materials include zeolite A, zeolite L, zeolite beta, zeolite X, zeolite Y, ZSM-5, MCM-22, and MCM-41. In a more specific embodiment, the zeolite is an X-type zeolite.

A catalyst comprising a substrate that supports a promoting metal or a combination of metals can be used to catalyze the reaction of hydrocarbons. The method of preparing the catalyst, pretreatment of the catalyst, and reaction conditions can influence the conversion, selectivity, and yield of the reactions.

The various elements that make up the catalyst can be derived from any suitable source, such as in their elemental form, or in compounds or coordination complexes of an organic or inorganic nature, such as carbonates, oxides, hydroxides, nitrates, acetates, chlorides, phosphates, sulfides and sulfonates. The elements and/or compounds can be prepared by any suitable method, known in the art, for the preparation of such materials.

The term “substrate” as used herein is not meant to indicate that this component is necessarily inactive, while the other metals and/or promoters are the active species. On the contrary, the substrate can be an active part of the catalyst. The term “substrate” would merely imply that the substrate makes up a significant quantity, generally 10% or more by weight, of the entire catalyst. The promoters individually can range from 0.01% to 60% by weight of the catalyst, optionally from 0.01% to 50%. If more than one promoters are combined, they together generally can range from 0.01% up to 70% by weight of the catalyst. The elements of the catalyst composition can be provided from any suitable source, such as in its elemental form, as a salt, as a coordination compound, etc.

The addition of a support material to improve the catalyst physical properties is possible. Binder material, extrusion aids or other additives can be added to the catalyst composition or the final catalyst composition can be added to a structured material that provides a support structure. Such components may also increase the macroporosity and the physical strength of the final catalyst form. The combination of the catalyst disclosed herein with additional elements such as a binder, extrusion aid, structured material, or other additives, and their respective calcination products, are included within the scope of the invention.

All suitable methods for catalyst preparation should be considered as applicable. Particularly effective techniques are those utilized for the preparation of solid catalysts. Conventional methods include co-precipitation from an aqueous, an organic or a combination solution-dispersion, impregnation, dry mixing, wet mixing or the like, alone or in various combinations. In general, any method can be used which provides compositions of matter containing the prescribed components in effective amounts. According to an embodiment the substrate is charged with promoter via an incipient wetness impregnation. Other impregnation techniques such as by soaking, pore volume impregnation, or percolation can optionally be used. Alternate methods such as ion exchange, wash coat, precipitation, and gel formation can also be used. Various methods and procedures for catalyst preparation are listed in the technical report Manual of Methods and Procedures for Catalyst Characterization by J. Haber, J. H. Block and B. Dolmon, published in the International Union of Pure and Applied Chemistry, Volume 67, Nos. 8/9, pp. 1257-1306, 1995, incorporated herein in its entirety.

Embodiments of the particular reactor system may be determined based on the particular design conditions and throughput, as will be understood by one of ordinary skill in the art, and are not meant to be limiting on the scope of the present invention.

The term “conversion” refers to the percentage of reactant (e.g. toluene) that undergoes a chemical reaction.

The term “molecular sieve” refers to a material having a fixed, open-network structure, usually crystalline, that may be used to separate hydrocarbons or other mixtures by selective occlusion of one or more of the constituents, or may be used as a catalyst in a catalytic conversion process.

Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

The term “regenerated catalyst” refers to a catalyst that has regained enough activity to be efficient in a specified process. Such efficiency is determined by individual process parameters.

The term “selectivity” refers to the relative activity of a catalyst in reference to a particular compound in a mixture. Selectivity is quantified as the proportion of a particular product relative to all other products.

The term “zeolite” refers to a molecular sieve containing a silicate lattice, usually in association with some aluminum, boron, gallium, iron, and/or titanium, for example. In the following discussion and throughout this disclosure, the terms molecular sieve and zeolite will be used more or less interchangeably. One skilled in the art will recognize that the teachings relating to zeolites are also applicable to the more general class of materials called molecular sieves.

The various embodiments of the present invention can be joined in combination with other embodiments of the invention and the listed embodiments herein are not meant to limit the invention. All combinations of various embodiments of the invention are enabled, even if not given in a particular example herein.

While illustrative embodiments have been depicted and described, modifications thereof can be made by one skilled in the art without departing from the spirit and scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.).

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Also, it is within the scope of this disclosure that the embodiments disclosed herein are usable and combinable with every other embodiment disclosed herein, and consequently, this disclosure is enabling for any and all combinations of the embodiments disclosed herein. Other and further embodiments, versions and examples of the invention may be devised without departing from the basic scope thereof and the scope thereof is determined by the claims that follow.

What is claimed is:

1. A method for producing styrene comprising:
reacting toluene with a C₁ source in a reactor in the presence of a catalyst to form a product stream comprising ethylbenzene and styrene, the reactor being sized and configured to provide for at least one moving catalyst bed therethrough;
wherein the C₁ source is selected from the group consisting of methanol, formaldehyde, formalin, trioxane, methylformcel, paraformaldehyde, methylal, dimethyl ether, and combinations thereof.

2. The method of claim 1, wherein the catalyst time on stream prior to regeneration is less than 24 hours.

3. The method of claim 1, wherein the contact time of the reactants with the catalyst in the reactor is less than 10 seconds.

4. The method of claim 1, wherein the reactor comprises:
a shell defining an interior cavity; and
at least one interior member defining a plurality of member openings comprising at least one particulate opening and at least one fluid opening;
wherein the interior member is disposed within the interior cavity and sized and configured to temporarily retain at least a portion of the catalyst and to permit at least a portion of a feedstream comprising the toluene and C₁ source to flow through the fluid opening and react over the catalyst, thereby forming at least one spent catalyst.

5. The method of claim 4, wherein the particulate opening is sized and configured to allow a flow of catalyst therethrough and the fluid opening is sized and configured to allow the feedstream to flow therethrough and to create turbulence in the feedstream flowing therethrough such that the turbulent feedstream agitates and displaces at least a portion of the temporarily retained catalyst.

6. The method of claim 1, wherein the catalyst is based on a zeolite selected from the group consisting of faujasites.

7. The method of claim 6, wherein the catalyst is based on an X-type zeolite.

8. The method of claim 6, wherein the catalyst is promoted with a promoter selected from the group consisting of Ru, Rh, Ni, Co, Pd, Pt, Mn, Ti, Zr, V, Nb, K, Cs, Ga, B, P, Rb, Ag, Ge, Cu, Mg, Fe, Mo, Ce, and Na and combinations thereof.

9. The method of claim 5, further comprising:
withdrawing the product stream and spent catalyst from the reactor;
regenerating the spent catalyst in a regeneration vessel to produce regenerated catalyst; and
recycling the regenerated catalyst to the reactor.

10. The method of claim 9, wherein the catalyst time on stream prior to regeneration is less than about 24 hours and the average contact time of the reactants with the catalyst is less than 10 seconds.

11. A method for producing styrene comprising:
reacting toluene with a C₁ source in a reactor in the presence of a catalyst to form a product stream comprising ethylbenzene and styrene, the reactor comprising:
a shell defining an interior cavity; and
at least one interior member defining a plurality of member openings, the interior member disposed within the interior cavity and sized and configured to temporarily retain at least a portion of the catalyst and to permit at least a portion of a feedstream comprising the toluene and C₁ source to flow through the member openings and react over the catalyst;
wherein the C₁ source is selected from the group consisting of methanol, formaldehyde, formalin, trioxane, methylformcel, paraformaldehyde, methylal, dimethyl ether, and combinations thereof.

12. The method of claim 11, wherein the interior member is a tray orientated substantially perpendicular to the longitudinal axis of the reactor.

13. The method of claim 11, wherein the catalyst time on stream prior to regeneration is less than 24 hours.

14. The method of claim 11, wherein the contact time of the reactants with the catalyst in the reactor is less than 10 seconds.

15. The method of claim 11, wherein the average contact time of the reactants and catalyst is less than 3 seconds.

16. The method of claim 11, wherein the catalyst is based on a zeolite selected from the group consisting of faujasites.

17. The method of claim 16, wherein the catalyst is based on an X-type zeolite.

18. The method of claim 16, wherein the catalyst is promoted with a promoter selected from the group consisting of Ru, Rh, Ni, Co, Pd, Pt, Mn, Ti, Zr, V, Nb, K, Cs, Ga, B, P, Rb, Ag, Ge, Cu, Mg, Fe, Mo, Ce, and Na and combinations thereof.

19. A method for making styrene comprising:
reacting toluene with a C₁ in a reactor over a catalyst to form a product stream comprising ethylbenzene and styrene, the reactor comprising:
a shell defining an interior cavity; and
at least one interior member defining a plurality of member openings, the interior member disposed within the interior cavity and sized and configured to temporarily retain at least a portion of the catalyst and to permit at least a portion of a feedstream comprising the toluene and C₁ source to flow through the member openings and react over the catalyst, thereby forming at least one spent catalyst;
withdrawing the product stream and spent catalyst from the reactor;
regenerating the spent catalyst in a regeneration vessel to produce regenerated catalyst; and
recycling the regenerated catalyst to the reactor.

20. The method of claim 19, wherein the catalyst time on stream prior to regeneration is less than about 24 hours and the average contact time of the reactants with the catalyst is less than about 10 seconds.
